(19) United States
(12) Patent Application Publication
(10) Pub. No.: US 2010/0228068 A1
(43) Pub. Date: Sep. 9, 2010

(54) PRODUCTION OF LINEAR ALKANES BY HYDROTREATING MIXTURES OF TRIGLYCERIDES WITH VACUUM GASOIL

(75) Inventors: Paul O'Connor, Hoevelaken (NL); Avelino Corma Camos, Valencia (ES); Dennis Stamires, Danapoint, CA (US)

Correspondence Address: HOWREY LLP-EU, C/O IP DOCKETING DEPARTMENT, 2941 FAIRVIEW PARK DR., SUITE 200, FALLS CHURCH, VA, 22042, US

(73) Assignee: BIOECON INTERNATIONAL HOLDING N.V., CURACAO (NL)

(21) Appl. No.: 12/377,389

(22) PCT Filed: Aug. 15, 2007
(86) PCT No.: PCT/EP2007/058468

§ 371 Date: Feb. 13, 2009

(30) Foreign Application Priority Data
Aug. 16, 2006 (US) 60837932
Nov. 14, 2006 (US) 60858709

(51) Int. Cl. C07C 1/20

(52) U.S. Cl. 585/733

(57) ABSTRACT

A process is disclosed for mild hydro-conversion of oxygenated hydrocarbon compounds. The oxygenated hydrocarbon compounds are contacted with a hydro-conversion catalyst material at a reaction pressure below 100 bar.

Preferred oxygenated hydrocarbon compounds are those obtained by the liquefaction of biomass.

In a specific embodiment the process is used for production of normal alkanes by hydrotreating mixtures of triglycerides (or compounds derived-from triglycerides, including free fatty acids) and vacuum gasoil.

### CROSS REFERENCE TO RELATED APPLICATION

This application is a continuation of International Application No. PCT/EP 2007/058468, filed on Aug. 15, 2007, which was published under PCT Article 21(2) in English, the contents of which are hereby incorporated by reference, in their entirety.

### BACKGROUND OF THE INVENTION

1. Field of the Invention

The invention relates to a process for production of alkanes, alcohols, olefins, and other components with a higher hydrogen to carbon ratio, from oxygenated compounds, such as glycerol, carbohydrates, sugar alcohols or other oxygenated biomass-derived molecules such as starches, cellulose, and hemicellulose-derived compounds, optionally mixed with petroleum derived feedstocks, in a mild hydroconversion process .

In a specific embodiment this invention relates to a process for production of alkanes by hydrotreating mixtures of triglycerides with vacuum gas-oil.

2. Description of the Related Art

Declining petroleum resources, combined with increased demand for petroleum by emerging economies, as well as political and environmental concerns about fossil fuels, are causing society to search for new sources of liquid fuels. In this respect, plant biomass is the only current sustainable source of organic carbon, and biofuels, fuels derived from plant biomass, are the only current sustainable source of liquid fuels (Klass 2004; Wyman, Decker et al. 2005). Biofuels have significantly less greenhouse gas emissions than fossil fuels, and can even be greenhouse gas neutral if efficient methods for biofuels production are developed (Lynd, Cushman et al. 1991; Wyman 1994). Vegetable oils, which consist of triglyercies, are one of the most promising feedstocks for biofuels production (Huber, Iborra et al. In Press). Inexpensive triglycerides sources, such as yellow (waste restaurant oil) and trap (which are collected at wastewater treatment plants) greases, can also be used as feedstocks for fuel production (Schumacher, Gerpen et al. 2004). Vegetable oils can be used directly in diesel engines, however there are a number of disadvantages of pure vegetable oils including: high viscosity, low volatility, and engine problems (including coking on the injectors, carbon deposits, oil ring sticking, and thickening of lubricating oils) (Ma and Hanna 1999; Knothe, Krahl et al. 2005). These problems require that vegetable oils be upgraded if they are to be used as a fuel in standard diesel engines.

The most common way of upgrading vegetable oils is by transesterification into alkyl-fatty esters (bio-diesel). The economics of biodiesel production depend heavily upon the price of co-product glycerol. As biodiesel production increases, the price of glycerol is projected to significantly drop, and the price of glycerol has already dropped by almost half over the last few years (McCoy 2005). The decrease in the price of glycerol would cause the production price of biodiesel to increase.

Another option for biofuels production is to use biomass-derived feedstocks in a petroleum refinery. Petroleum refineries are already built and using this existing infrastructure for biofuels production would require little capital cost investment. The European Commission has set a goal that by 2010, 5.75% of transportation fuels in the EU will be biofuels, and co-feeding biomass-derived molecules into a petroleum refinery could rapidly decrease our dependence on petroleum feedstocks. Hydrotreating is a common process used in the petroleum refinery, and is mainly used to remove S, N₂ and metals from petroleum derived feedstocks (Farrauto and Bartholomew 1997).

In 1991 Craig and Soveren patented a process to produce liquid paraffins (mainly normal C₁₅-C₁₈ alkanes) by hydrotreating of vegetable oils including canola oil, sunflower oil, soy bean oil, rapeseed oil, palm oil, fatty acid fraction of tall oil, and mixtures of the above compounds (Craig and Soveran 1991). In their patent they disclosed the production of a diesel fuel additive that was high in C₁₅-C₁₈ alkanes. These normal alkanes have a high cetane number (above 98), whereas typical diesel fuel has a cetane number around 45. Craig and Soveran recommend that the alkanes produced by hydrotreating of vegetable oils be mixed with diesel fuel in the range of 5-30% by volume. They claim a process for hydroprocessing of vegetable oils at a temperature of from 350 to 450° C., H₂ partial pressure 48-152 bar, and a liquid-hourly space velocity (LHSV) of 0.5-5.0 hr⁻¹. The catalysts they disclose are typical commercial hydroprocessing catalysts including cobalt-molybdenum (Co—Mo), nickel molybdenum (Ni Mo) or other transition metal based hydroprocessing catalysts.

In 1998 another patent appeared for hydroprocessing of tall oil, vegetable oil, animal fats or wood oils by Monnier et al. using standard hydroprocessing catalysts, temperatures of from 350 to 370° C., H₂ partial pressure of 40-150 bar, and a LHSV of 0.5-5.0 hr⁻¹ (Monnier, Tourigny et al. 1998). Tall oil is a by-product in the Kraft pulping of pine and spruce trees, which can have very little economic value. Tall oil contains large amount of unsaturated fatty acids (30-60 wt %). Alkanes were produced from hydrotreating of tall oil, and a ten month on-road test of six postal delivery vans showed that engine fuel economy was greatly improved by a blend of petrodiesel with hydrotreated tall oil (Stumborg, Wong et al. 1996). According to Stumborg et al. the advantages of hydrotreating over trans-esterification are that it has lower processing cost (50% that of transesterification), compatibility with current infrastructure, engine compatibility, and feedstock flexibility (Stumborg, Wong et al. 1996).

In a typical petroleum refinery hydrotreating is done with vacuum-gas oil. The objective of hydrotreating in a petroleum refinery is to remove sulfur (Hydro-desulfurization, HDS), nitrogen (Hydrodenitrogenation, HDN), metals (hydrodemetalation, HDM), and oxygen (hydrodeoxygenation, HDO) from the heavy gas oil feedstock. Hydrogen is added with the heavy gas oil feed. Typical catalysts used for hydrotreating include sulfided CoMo and NiMo. Typical reaction conditions include temperatures of from 300 to 450° C., 35-170 bar H₂ partial pressure, and LHSV of from 0.2 to 10 h⁻¹.

Oxygenated hydrocarbon compounds, such as bio-oils obtained in the liquefaction of biomass, or glycerol as obtained in the transesterification of triglycerides in bio-diesel production processes, do not normally contain significant amounts of aromatics, sulfur compounds, or nitrogen compounds. Accordingly, there is no need to treat these materials in HDS, HDN, or HDA processes.

Chen et al. report the major challenge with biomass conversion to be the removal of oxygen from the biomass and enriching the hydrogen content of the hydrocarbon product. They define the effective hydrogen to carbon ratio (H/C_{eff}) defined in Equation 1. The H/C_{eff} ratio of biomass derived-oxygenated hydrocarbon compounds is lower than petroleum-derived feedstocks due to the high oxygen content of biomass-derived molecules. The H/C_{eff} ratio of carbohydrates, sorbitol and glycerol (all biomass-derived compounds) are 0, ⅓ and ⅔ respectively. The H/C_{eff} ratio of petroleum-derived feeds ranges from 2 (for liquid alkanes) to 1 (for benzene). In this respect, biomass can be viewed as a hydrogen deficient molecule when compared to petroleum-based feedstocks.

$$H / {C}_{eff} = \frac{H - 2 O - 3 N - 2 S}{C} \qquad ( 1 )$$

where H, C, O, N and S are the moles of hydrogen, carbon, oxygen, nitrogen and sulfur respectively.

Glycerol is currently a valuable by-product of biodiesel production, which involves the transesterification of triglycerides to the corresponding methyl or ethyl esters. As biodiesel production increases, the price of glycerol is projected to drop significantly. In fact, the price of glycerol has already dropped by almost half over the last few years. [McCoy, 2005 #6] Therefore it is desirable to develop inexpensive processes for the conversion of glycerol into chemicals and fuels.

Methods for conversion of solid biomass into liquids by acid hydrolysis, pyrolysis, and liquefaction are well known [Klass, 1998 #12]. Solid materials including lignin, humic acid, and coke are byproducts of the above reaction. A wide range of products are produced from the above reactions including: cellulose, hemicellulose, lignin, polysaccharides, monosaccharides (e.g. glucose, xylose, galatose), furfural, polysaccharides, and lignin derived alcohols (coumaryl, coniferyl and sinapyl alcohols).

The object of the present invention is to provide a process for improving the H/C_{eff} ratio of oxygenated hydrocarbon compounds. It is a further object of the present invention to provide such a process that makes optimum use of existing refinery equipment and existing hydroconversion catalysts. It is yet another object of the present invention to provide a process that can be carried out under mild conditions of pressure and temperature so as to minimize equipment cost and undesirable side reactions.

A specific object of the present invention is to provide a process for co-treating vacuum gas oil and vegetable oil

### SUMMARY OF THE INVENTION

The invention relates generally to a process for the mild hydroconversion of oxygenated hydrocarbon compounds, comprising the step of contacting a reaction feed comprising an oxygenated hydrocarbon compound with a hydroconversion catalyst material at a reaction pressure below 100 bar.

In a specific embodiment the invention relates to a process for production of normal alkanes by hydrotreating mixtures of triglycerides (or compounds derived—from triglycerides, including free fatty acids) and vacuum gasoil. The mixtures are 99.5 to 50 wt % vacuum gasoil, with the remainder of the feedstock being triglycerides, or triglyceride-derived molecules such as diglycerides, monoglyceries and free-fatty acids. The triglycerides may include sunflower oil, rapeseed oil, soybean oil, canola oil, waste vegetable oil (yellow grease), animal fats, or trap grease. Tall oil or other biomass derived oils, containing mixtures of free fatty acids and triglycerides can also be used for the hydrotreating process. The catalysts that can be used include sulfided NiMo/Al₂O₃, CoMo/Al₂O₃ or other standard hydrotreating catalysts known to those skilled in the art. The hydrotreating reaction conditions include temperatures from 300 to 450° C., inlet H₂ partial pressures of 35 to 200 bar, and LHSV of 0.2 to 15 h⁻¹.

### BRIEF DESCRIPTION OF THE DRAWINGS

FIG. 1 shows the reaction mechanism for conversion of triglycerides.

FIG. 2 represents sulfur conversion for hydrotreating of vegetable oil-heavy gas oil feeds.

FIG. 3. represents the nitrogen conversion for hydrotreating of vegetable oil-heavy gas oil feeds.

FIG. 4. shows simulated distillation yields for hydrotreating of vegetable oil-heavy gas oil feeds.

FIG. 5. shows normal alkane, CO, CO₂, and propane yields for hydrotreating of vegetable oil-heavy gas oil feeds.

FIG. 6. shows the percentage of normal C₁₅ to C₁₈ alkanes in a 250 to 380° C. simulated distillation fraction as a function of hydrotreating temperature and percentage of vegetable oil in vacuum gasoil.

FIG. 7. shows the percentage of maximum theoretical yields of n-C₁₅-C₁₈ alkanes for hydrotreating of vegetable oil-heavy gas oil feeds.

### DETAILED DESCRIPTION OF THE INVENTION

This invention generally relates to a process for mild hydroconversion of oxygenated hydrocarbon compounds, comprising the step of contacting a reaction feed comprising an oxygenated hydrocarbon compound with a hydroconversion catalyst material at a reaction pressure of less than 100 bar. In a preferred embodiment the reaction pressure is less than 40 bar.

This invention more specifically relates to a process for the hydroconversion of glycerol, carbohydrates, sugar alcohols or other biomass derived oxygenated compounds such as starches, cellulose-derived compounds, and hemicellulose-derived compounds. In a preferred embodiment these compounds are co-fed with petroleum derived feedstocks in a standard or modified hydroconversion process. Mixtures of oxygenated compounds, such as those found in bio-oils derived from pyrolysis or liquefaction, are also included in the definition of biomass-derived oxygenated compound. In general, oxygenated hydrocarbon compounds that have been produced via the liquefaction of a solid biomass material are particularly preferred. In a specific embodiment the oxygenated hydrocarbon compounds are produced via a mild hydrothermal conversion process, such as described in co-pending application EP 061135646, filed on May 5, 2006, the disclosures of which are incorporated herein by reference. In an alternate specific embodiment the oxygenated hydrocarbon compounds are produced via a mild pyrolysis process, such as described in co-pending application EP 061135679, filed on May 5, 2006, the disclosures of which are incorporated herein by reference.

The oxygenated hydrocarbon compounds may be mixed with an inorganic material, for example as a result of the process by which they were obtained. In particular, solid biomass may have been treated with a particulate inorganic material in a process such as described in co-pending application EP 061135810, filed May 5, 2006, the disclosures of which are incorporated herein by reference. These materials may subsequently be liquefied in the process of EP 061135646 or that of EP 061135679, cited herein above. The resulting liquid products contain the inorganic particles. It is not necessary to remove the inorganic particles from the oxygenated hydrocarbon compounds prior to the use of these compounds in the process of the present invention. To the contrary, it may be advantageous to leave the inorganic particles in the oxygenated hydrocarbon feed, in particular if the inorganic material is a catalytically active material. In the alternative the inorganic material may be used as a catalyst carrier.

Similarly, the oxygenated hydrocarbon compounds may have been obtained by liquefaction of a biomass material comprising an organic fiber, as disclosed in co-pending application EP 06117217.7, filed Jul. 14, 2006, the disclosures of which are incorporated herein by reference. In this case the oxygenated hydrocarbon compounds may contain organic fibers. It may be advantageous to leave these fibers in the reaction feed, as they may have catalytic activity. The fibers may also be used as a catalyst carrier, for example by bringing the fibers into contact with a metal.

In a specific embodiment the reaction feed further comprises a crude oil-derived material, for example vacuum gas-oil. Crude oil-derived materials are generally less reactive than oxygenated hydrocarbon compounds. For this reason it is preferred to use a continuous process, and to inject the oxygenated compounds at a point downstream from the injection point of the crude oil-derived compounds, to ensure a shorter contact time of the former with the hydroconversion catalyst material.

It has been found that the reaction feed may comprise some amounts of water. This is particularly advantageous, because feedstocks such as bio-oil and glycerol derived from biomass conversion processes tend to be mixed with water.

The process according to the invention can be carried out in a fixed bed, in a moving bed, or in an ebullating bed. Carrying out the process in an ebullating bed is particularly preferred. It is possible to carry out the reaction in a conventional hydro-processing reactor.

The process according to the invention can be carried out in a single reactor or in multiple reactors. If multiple reactors are used, the catalyst mixture used in the two reactors may be the same or different. If two reactors are used, one may or may not perform one or more of: intermediate phase separation, stripping, H₂ quenching, etc. between the two stages.

The process conditions for a preferred embodiment of the process according to the invention may be as follows. The temperature generally is 200-500° C., preferably 300-400° C. The pressure generally is in the range of 20-100 bar, preferably less than 40 bar The liquid hourly space velocity generally is 0.1-3 h⁻¹, preferably 0.3-2 h⁻¹. The hydrogen to feed ratio generally is 300-1,500 NI/I, preferably less than 600 NI/I. The process is carried out in the liquid phase.

Any conventional hydroprocessing or hydroconversion catalyst as used in oil refining is suitable for use in the process of the present invention. Suitable examples include bimetallic catalysts comprising a metal from Group VIB and a metal from Group VIIIB of the Periodic Table of the Elements. The Group VIIIB metal preferably is a non-noble metal. Examples include Co/Mo, Ni/W, Co/W catalysts.

For hydrodesulfurization it is in general advantageous to pre-sulfide the catalyst. Pre-sulfidization is in general not required for the hydroconversion of oxygenated hydrocarbons.

In the alternative the hydroconversion catalyst material comprises a basic material. Examples of suitable basic materials include layered materials, and materials obtained by heat-treating layered materials. Preferably the layered materials are selected from the group consisting of smectites, anionic clays, layered hydroxy salts, and mixtures thereof. Hydrotalcite-like materials, in particular Mg—Al, Mg—Fe, and Ca—Al anionic clays, are particularly preferred. It has surprisingly been found that basic materials are also suitable for the hydro-processing of a crude-oil derived material, such as VGO, as may be used as a first feedstock in certain embodiments of the process of the present invention.

Preferably, the particles also contain metals like W, Mo, Ni, Co, Fe, V, and/or Ce. Such metals may introduce a hydrotreating function into the particles (especially W, Mo, Ni, Co, and Fe) or enhance the removal of sulfur- and/or nitrogen-containing species (Zn, Ce, V).

The basic catalytic materials may be used as such, or may be used in admixture with a conventional hydro-processing catalyst.

The empirical formula of cellulose is (C₆H₁₀O₅)ₙ. Chemically cellulose is a polymer of glucose, which has the empirical formula C₆H₁₂O₆. Both cellulose and glucose have a H/C_{eff} ratio of 0. Although it might be desirable to fully convert cellulose to alkanes, it is not necessary to fully hydrogenate cellulose, or oxygenated hydrocarbon compounds derived from cellulose, in order to obtain useful liquid fuels. In many cases partial hydrogenation is sufficient, and more desirable from the perspective of hydrogen consumption. The hydro-conversion reaction is considered successful if it results in an increase of the H/C_{eff} ratio by about 0.2, for example from 0 to 0.2 (in the case of cellulose or glucose), or from 0.3 to 0.5 in the case of glycerol. Accordingly, the molar ratio of hydrogen in the reaction mixture to oxygen in the oxygenated hydrocarbon feed suitably is in the range of from 0.1 to 0.3.

In a specific embodiment this invention relates to a process for the production of normal alkanes by hydrotreating mixtures of triglycerides (or compounds derived-from triglycerides including free fatty acids) and vacuum gasoil. The mixtures are 99.5 to 50.0 wt % vacuum gasoil with the remainder of the feedstock being triglycerides or triglyceride-derived molecules such as diglycerides, monoglyceries and free-fatty acids. The triglycerides can include sunflower oil, rapeseed oil, soybean oil, canola oil, waste vegetable oil (yellow grease), animal fats, or trap grease. Tall oil or other biomass derived oils, containing mixtures of fatty acids and triglycerides can also be used for the hydrotreating process. The catalysts that can be used include sulfided NiMo/Al₂O₃, CoMo/Al₂O₃ or other standard hydrotreating catalysts known to those skilled in the art. The hydrotreating reaction conditions include temperatures from 300 to 450° C., inlet H₂ partial pressures of 35 to 200 bar, and LHSV values of 0.2-15 h⁻¹.

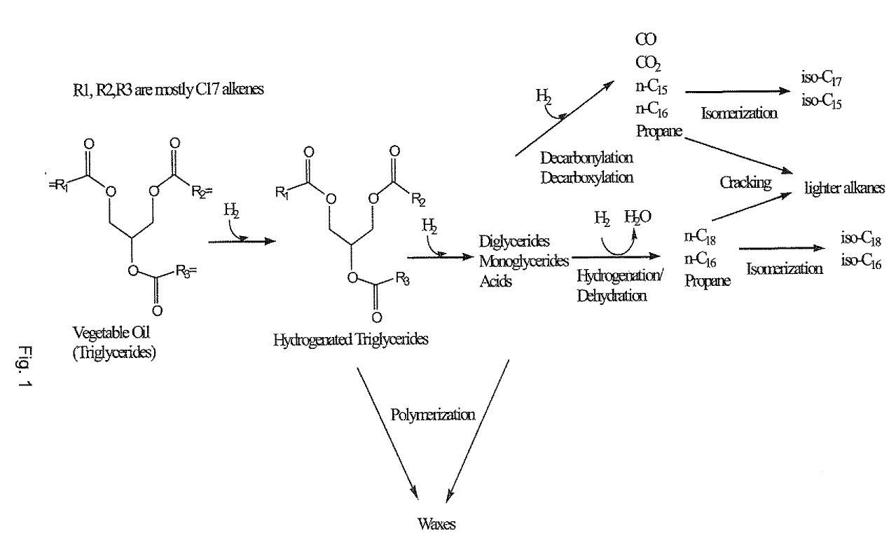

During the hydrotreating of vegetable oils the C═C bonds of the vegetable oils are first hydrogenated as shown in FIG. 1. The hydrogenated vegetable oils then form free fatty acids, diglyerides and monoglycerides. Operation at low temperature and high space velocities will cause the hydrogenated vegetable oils and products derived from the hydrogenated vegetable oils to form waxes. These waxes could plug the reactor. The free fatty acids, diglycerides, monoglycerides and triglycerides undergo two different pathways to produce normal alkanes. The first is decarbonylation, which produces normal liquid alkanes (C₁₇ if from a C₁₈ free fatty acid), CO or CO₂, and propane. Alternatively, these feeds may undergo a dehydration/hydrogenation pathway to produce a normal liquid alkane (C₁₈ if from a C₁₈ acid) and propane. The liquid normal alkanes produced undergo isomerization and cracking to produce less valuable lighter and isomerized alkanes. These alkanes are less valuable for diesel fuel usage because they have a lower cetane number. The isomerization and cracking reactions are a function of the reaction temperature, and the concentration of vegetable oil in the vegetable oil-vacuum gasoil mixture, as we will show in this patent. The fractions coming from the hydrotreating reactor can then be separated by distillation.

### EXAMPLES

The following Examples are included solely to provide a more complete disclosure of the subject invention. Thus, the following Examples serve to illustrate the nature of the invention, but do not limit the scope of the invention disclosed and claimed herein in any fashion.

Experiments described in this patent were performed in a fixed bed hydrotreating reactor. The catalyst (NiMo/Al₂O₃, Haldor-Topsoe XXX) was loaded into a stainless steel tubular reactor (2.54-cm I.D. and 65 cm in length). The catalysts were pre-sulfided using a mixture of H₂S/H₂ (9 vol % H₂S) at atmospheric pressure and 400° C. for 9 h. The reaction conditions for these examples were as follows: temperatures 300 to 450° C., pressures 50 bar, LHSV 4.97 h⁻¹, and H₂-to-feed ratio of 1600 ml H₂ gas/ml liquid feed. The gas inlet was 91% H₂ with the balance being Ar, which was used as an internal standard.

Vacuum gasoil (VGO) was obtained from the Huelva refinery (CEPSA group). The VGO feed had a carbon content of 88 weight %. The carbon yields are defined as the moles of carbon in each product divided by the carbon in the feed. Sunflower oil (Califour brand) was purchased for mixing with vacuum gasoil.

The reaction gases were analyzed using a Varian 3800-GC equipped with three detectors, a Thermal Conductivity Detector (TCD) for analysis of H₂ and N₂, which were separated in a 15 m molecular sieve column, and a Flame Ionization Detector (FID) for C₁ to C₆ hydrocarbons separated in a 30 m Plot/Al₂O₃ column. Liquids samples were analyzed for normal alkane content with a Varian 3900-GC chromatograph equipped with a Petrocol-100 fused silica column connected to a FID detector following PIONA procedure. In addition, simulated distillation of vacuum gas oil (VGO) cracking samples were carried out using a Varian 3800GC chromatograph according to the ASTM-2887-D86 procedure. The concentrations of sulfur and nitrogen in the original feed and liquid products were determined by elemental analysis in a Fisons 1108 CHNS-O instrument.

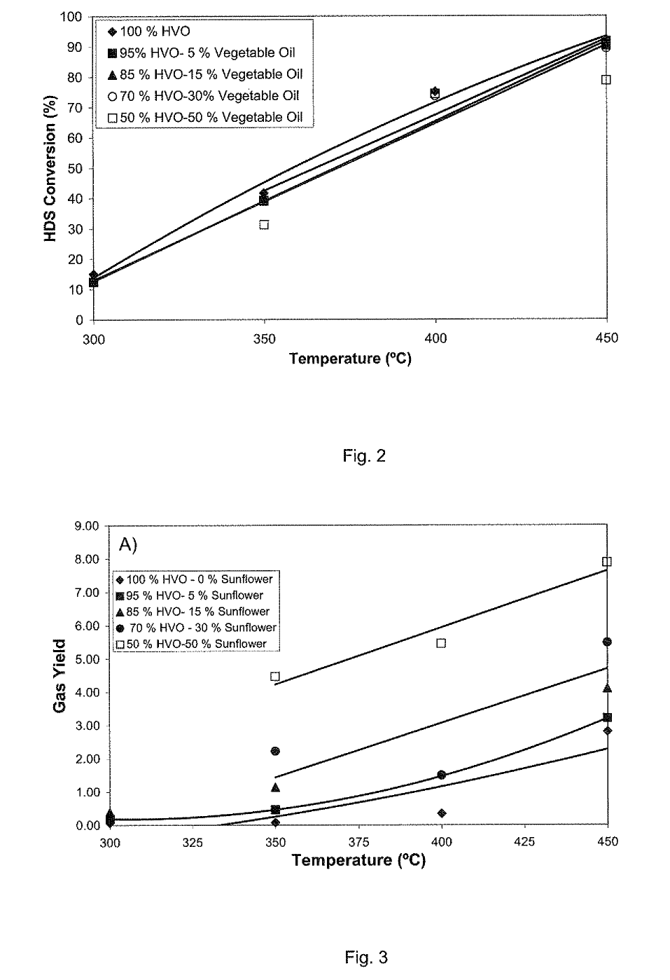

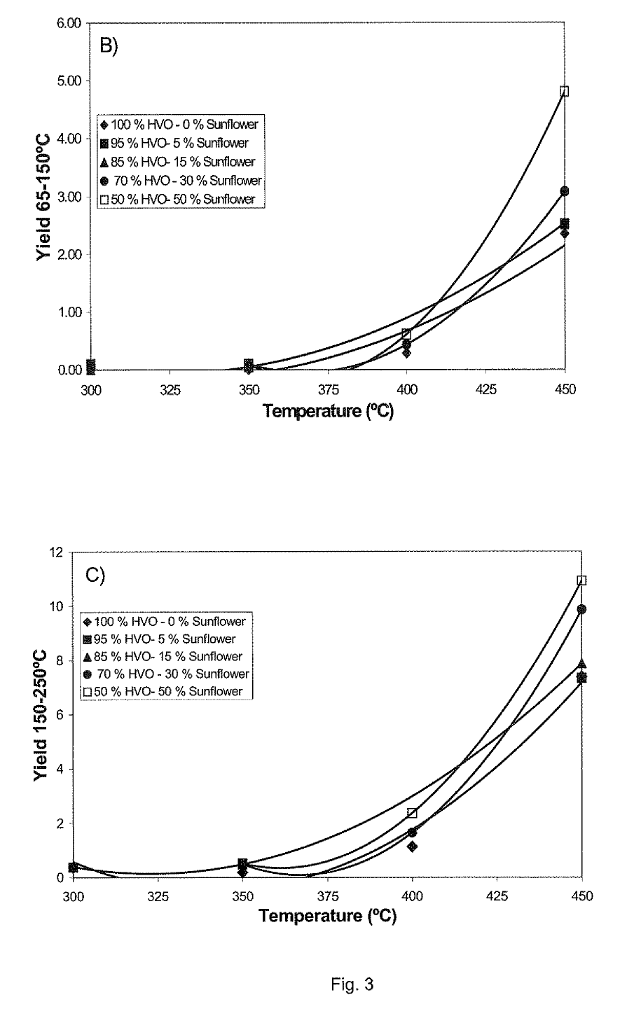

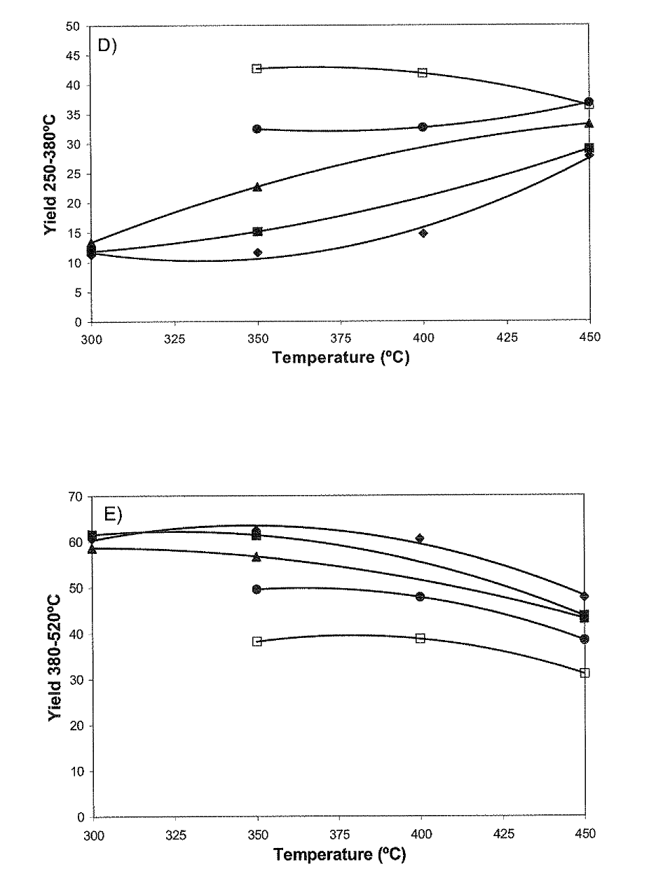

The following feeds were hydrotreated including: 100 wt % HVO, 95 wt % HVO-5 wt % Sunflower oil, 85 wt % HVO-15 wt % Sunflower oil, 70 wt % HVO-30 wt % Sunflower oil, and 50 wt % HVO-50 wt % Sunflower oil. The results for the hydro-desulfurization and hydrodenitrogenation are shown in FIGS. 2 and 3 respectively. As can be seen from these figures, mixing vegetable oils does not decrease the ability of the hydrotreating process to remove sulfur or nitrogen from the HVO feed.

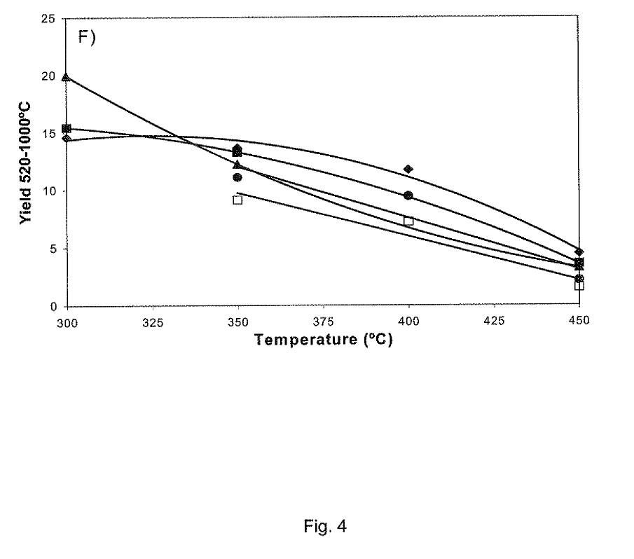

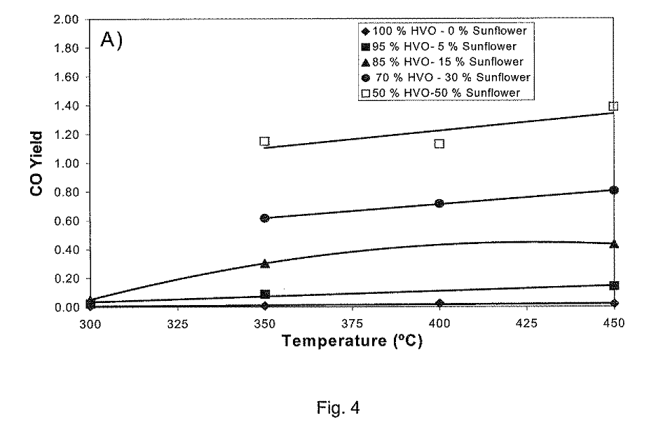

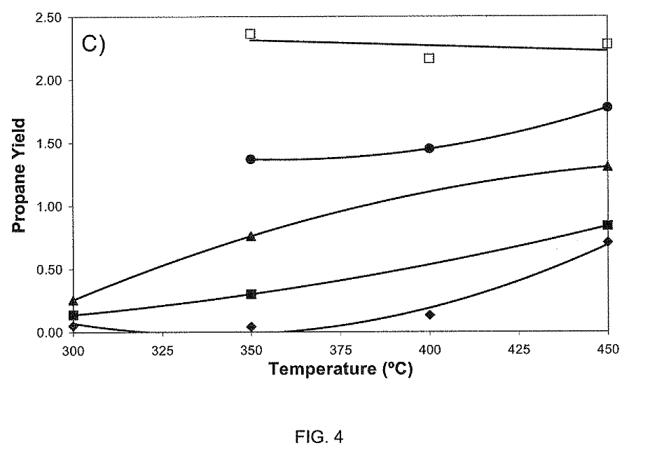

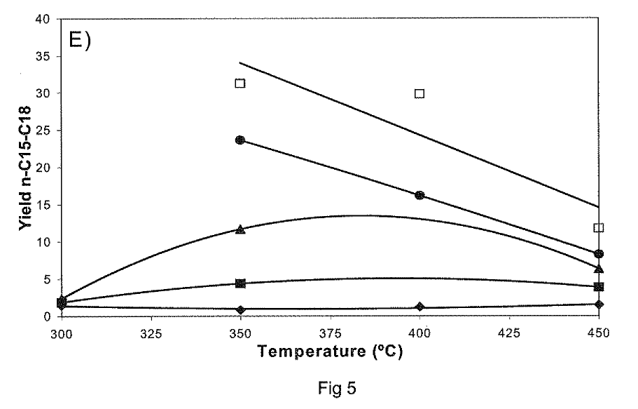

FIG. 4 shows the simulated distillation results for hydrotreating the different feeds. FIG. 5 shows the yields for the prominent alkanes, CO and CO₂ for the hydrotreating of different feeds. The gas yield increases as the concentration of sunflower oil increases (FIG. 4A). This is because propane, CO and CO₂ are formed during hydrotreating of triglyceride as shown in FIG. 5. The yields from the 380-520° C. and 520-1000° C. fractions decrease with both increasing concentration of sunflower oil and temperature. The yields of the 250 to 380° C. fraction (mainly diesel fuel) increases as the sunflower oil concentration increases. This fraction contains nC₁₅-nC₁₈ products, which are formed from the sunflower oil. The yield of nC₁₅-nC₁₈, shown in FIG. 5E, increases with increasing concentration of sunflower oil. For the feeds containing 30 wt % and 50 wt % sunflower oil the nC₁₅-nC₁₈ yields decrease when the reaction temperature is increased above 350° C. This is because the nC₁₅-nC₁₈ are cracked to lighter products at the higher temperature as shown by an increase in the 65-150° C. yield, 150-250° C. yield and the nC₈-nC₁₂ yield.

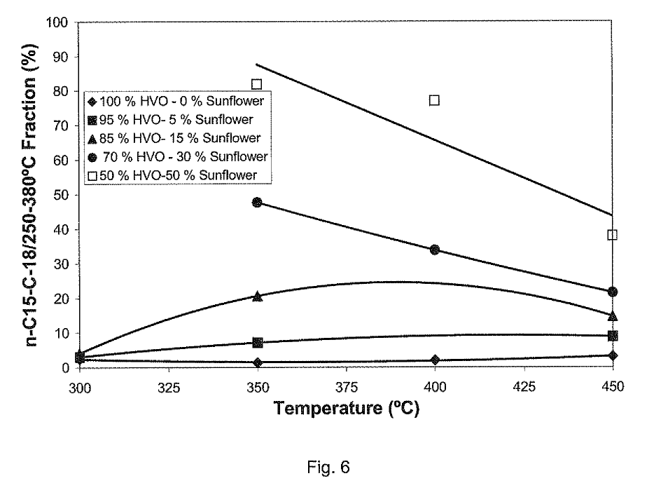

FIG. 6 shows the percentage of nC₁₅-nC₁₈ in the diesel fuel fraction (250-380° C.). This percentage increases as the sunflower concentration in the feed increases. The percentage also decreases as the temperature increases from 350 to 450° C. for the 30 wt % and 50 wt % sunflower oil feeds.

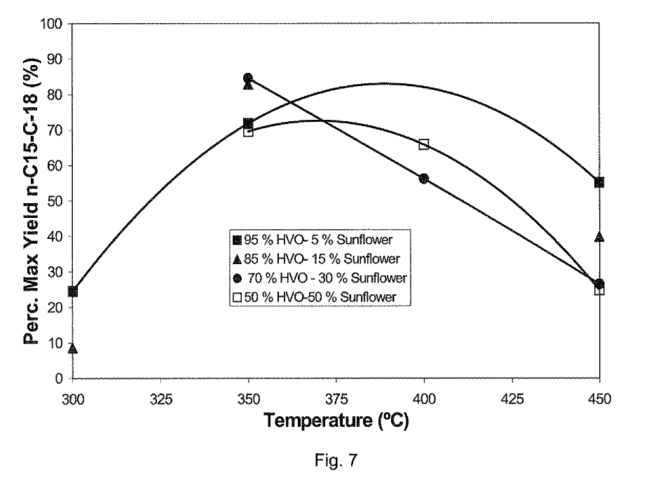

In FIG. 7 we show the percentage of maximum nC₁₅-nC₁₈ yield for the different HVO-Sunflower mixtures. The percentage of maximum nC₁₅-nC₁₈ yield (PMCY) is defined as the yield of nC₁₅-nC₁₈ minus the yield of nC₁₅-nC₁₈ from the HVO divided by the maximum nC₁₅-nC₁₈ yield if all of the fatty acids present in the triglyceride were converted into nC₁₅-nC₁₈. The PMCY increases as the temperature increases for the 5 wt % sunflower feed as shown in FIG. 7, and the PMCY for this feed is 65-70% at temperatures from 350 to 450° C. THE PMCY for the 15 wt % sunflower feed increases from 9 to 83% as the temperature increases from 300 to 350° C., while a further increase in the temperature to 450° C. decreases the PMCY to 40%. The PMCY for the 30 wt % sunflower feed decreases from 85% to 56% to 26% as the temperature increases from 350° C. to 400° C. and to 450° C. The PMCY for the 50 wt % sunflower feed decreases from 70 to 26% as the temperature increases from 350° C. to 450° C. Thus there is both an optimal temperature and vegetable oil concentration for obtaining optimum yields for the nC₁₅-nC₁₈.

### Works Cited

Craig, W. K. and D. W. Soveran (1991). Production of hydrocarbons with a relatively high cetane rating. U.S. Pat. No. 4,992,605. USA.

Farrauto, R. J. and C. Bartholomew (1997). Introduction to Industrial Catalytic Processes, Chapman & Hall, London, UK.

Huber, G. W., S. Iborra, et al. (In Press). “Synthesis of transportation fuels from biomass: chemistry, catalysts and engineering.” Chemial Reviews.

Klass, D. L. (2004). Biomass for Renewable Energy and Fuels. Encyclopedia of Energy, Volume 1. C. J. Cleveland, Elsevier.

Knothe, G., J. Krahl, et al. (2005). The Biodiesel Handbook. Champaign, Ill., AOCS Press.

Lynd, L. R., J. H. Cushman, et al. (1991). “Fuel ethanol from cellulosic biomass.” Science 251: 1318-23.

Ma, F. and M. A. Hanna (1999). “Biodiesel production: a review.” Bioresource Technology 70: 1-15.

McCoy, M. (2005). “An Unlikely Impact.” Chem. Eng. News. 83 (8): 19-20.

Monnier, J., G. Tourigny, et al. (1998). Conversion of biomass feedstock to diesel fuel additive. U.S. Pat. No. 5,705,722. USA, Natural Resources Canada.

Schumacher, L. G., J. V. Gerpen, et al. (2004). Biodiesel Fuels. Encyclopedia of Energy. C. J. Cleveland. London, Elsevier.

Stumborg, M., A. Wong, et al. (1996). “Hydroprocessed vegetable oils for diesel fuel improvement.” Bioresource Technology 56(1): 13-18.

Wyman, C. E. (1994). “Alternative fuels from biomass and their impact on carbon dioxide accumulation.” Appl. Biochem. Biotechnol. 45/46: 897-915.

Wyman, C. E., S. R. Decker, et al. (2005). Hydrolysis of cellulose and hemicellulose. Polysaccharides. S. Dumitriu. New York, N. Y, Marcel Dekker, Inc.

Thus, the invention has been described by reference to certain embodiments discussed above. It will be recognized that these embodiments are susceptible to various modifications and alternative forms well known to those of skill in the art.

What is claimed is:

1. A process for the mild hydroconversion of oxygenated hydrocarbon compounds, comprising the step of contacting a reaction feed comprising an oxygenated hydrocarbon compound with a hydroconversion catalyst material at a reaction pressure below 100 bar.

2. The process of claim 1 wherein the reaction pressure is below 40 bar.

3. The process of claim 1 wherein the oxygenated hydrocarbon compound is derived from a biomass material.

4. The process of claim 1 wherein the reaction feed further comprises water.

5. The process of claim 1 wherein the reaction feed further comprises a crude-oil derived material.

6. The process of claim 5 wherein the crude-oil derived material comprises vacuum gas oil.

7. The process of claim 1 wherein the oxygenated hydrocarbon compound comprises a material selected from the group consisting of polysaccharides, oligosaccharides, sugars, polyhydric alcohols; oligohydric alcohols, monohydric alcohols, carboxylic acids, and mixtures thereof.

8. The process of claim 7 wherein the oxygenated hydrocarbon compound comprises glycerol.

9. The process of claim 1 which is a hydro-processing which is carried out in a hydro-processing unit.

10. The process of claim 9 whereby the hydro-processing is carried out with a first feedstock of crude oil origin and a second feedstock comprising an oxygenated hydrocarbon compound, whereby the first feedstock is brought into the hydro-processing unit at a first point, and the second feedstock is brought into the hydro-processing unit at a second point, separate from the first point.

11. The process of claim 10 wherein the second point is upstream from the first point.

12. The process of claim 10 wherein the second point is downstream from the first point.

13. The process of claim 10, wherein the first feedstock comprises vacuum gas oil.

14. The process of claim 10 wherein the second feedstock comprises glycerol.

15. The process of claim 10 wherein the second feedstock further comprises water.

16. The process of claim 10 wherein the second feedstock comprises a glycerol/water mixture produced in a biodiesel transesterification process.

17. The process of claim 1 whereby the catalyst comprises a basic material.

18. The process of claim 17 wherein the basic material is a layered material, or a heat treated form thereof.

19. The process of claim 18 wherein the layered material is selected from the group consisting of smectites, anionic clays, layered hydroxy salts, and mixtures thereof.

20. The process according to claim 19 wherein the layered material is a Mg—Al, Mg—Fe or a Ca—Al anionic clay.

21. The process according to claim 17, wherein the catalytic material further comprises a conventional hydro-processing catalyst.

22. The process according to claim 1 wherein the oxygenated hydrocarbons have been produced via the liquefaction of solid biomass.

23. The process according to claim 1 wherein the oxygenated hydrocarbons have been produced via the liquefaction of solid biomass under mild conditions.

24. The process according to claim 1 wherein the reaction feed contains an inorganic material.

25. The process according to claim 1 wherein the reaction feed contains an organic fiber.

26. The process according to claim 24 wherein the inorganic material functions as a catalyst or a catalyst carrier.

27. The process according to claim 25 wherein the organic fiber functions as a catalyst or a catalyst carrier.

28. The process according to claim 25 wherein the organic fiber is in contact with a metal.

29. The process of claim 6 when used in the production of C₁₅ to C₂₂ alkanes, said process comprising the step of hydrotreating a mixture comprising from 0.1 to 50.0 wt. % of a fatty acid compound and from 99.9-50.0 wt % vacuum gasoil, at a temperature in the range of from 300 to 450° C., and a reactor inlet H₂ partial pressure of from 35 to 200 bar in the presence of a hydrotreating catalyst.

30. The process of claim 29 wherein the liquid hourly space velocity of the hydrotreating step is in the range of from 0.2 to 15 hr⁻¹.

31. The process of claim 29 wherein the fatty acid compound comprises a triglyceride.

32. The process of claim 31 wherein the triglyceride is selected from the group consisting of sunflower oil, rapeseed oil, canola oil, soybean oil, waste vegetable oil, brown grease, animal fat, and derivatives and mixtures thereof.

33. The process of claim 32 wherein the triglyceride comprises sunflower oil.

34. The process of claim 29 wherein the hydrotreating catalyst comprises Mo, W, or mixtures thereof.

35. The process of claim 29 wherein the catalyst comprises Ni, Co, or mixtures thereof.

36. The process of claim 29 wherein the catalyst is in a sulfided form.

37. The process of claim 29 wherein the catalyst is a Ni/Mo or a Co/Mo catalyst.

38. The process of claim 29 wherein the catalyst is sulfided Ni/Mo on alumina, or sulfided Co/Mo on alumina.
